(19) United States
(12) Patent Application Publication
(10) Pub. No.: US 2009/0247836 A1
(43) Pub. Date: Oct. 1, 2009

(54) Medical System and Method for Serving Users with a Chronic Disease or Health State

(75) Inventors: Stephen Richard Cole, Durham, NC (US); John Francis Petro, Raleigh, NC (US)

Correspondence Address: WARD AND SMITH, P.A., 1001 COLLEGE COURT, P.O. BOX 867, NEW BERN, NC, 28563-0867, US

(73) Assignee: CONFIDANT INC., Durham, NC (US)

(21) Appl. No.: 12/396,011

(22) Filed: Mar. 2, 2009

Related U.S. Application Data
(60) Provisional application No. 61/032,123, filed on Feb. 28, 2008.

(51) Int. Cl. A61B 5/0205; A61B 5/145

(52) U.S. Cl. 600/301; 600/365

(57) ABSTRACT

A system and method collects medical data from a patient and transmits the data to a backend system. The data is analyzed and a feedback message is generated and transmitted to at least the patient.

### CROSS REFERENCE TO RELATED APPLICATIONS

This Application claims priority to Provisional Application Ser. No. 61/032,123 filed Feb. 28, 2008, the disclosure of which is incorporated herein by reference in its entirety. This Application is also related to U.S. Provisional Application Ser. No. 60/673,686 filed Dec. 20, 2004 and regular U.S. application Ser. No. 11/312,156 filed Dec. 20, 2005, now published as US Publication Number US 2006/0212316. The disclosures of said referenced Applications are hereby incorporated herein by reference in their entirety.

### FIELD OF THE INVENTION

This invention relates to a system and method for helping patients with chronic disease to manage their disease on a daily basis. More particularly, the invention relates to such a system and method in which personality defined feedback is provided to improve user self-care and regimen compliance.

### BACKGROUND OF THE INVENTION

Medical personnel managing patients with chronic conditions, like diabetes, today have less and less time available to provide adequate monitoring and equipment. Attempts have been made to develop remote monitoring systems but they are generally complicated and require complex communication transmissions. The systems do not provide adequate feedback to encourage and train patients to take better care of themselves.

A number of companies are currently involved in some form of remote patient monitoring for disease management. None of these companies provide direct, automated feedback based on remotely measured data, and thus do not provide for sustained improved behavior of the patient.

One prior art system that has been developed is disclosed in the previously-referenced US Patent Publication Number US 2006/0212316. In accordance with that system, an integrated system and method provides a feedback loop to help maintain improved behavior leading to improved health. At least one medical device is provided such as a blood pressure monitor, blood glucose detector, etc. for detecting a specific user physical data and/or condition. An interface device is connected thereto which is capable of transmitting specific user physical data to a wireless cellular telephone. The telephone is programmed to receive the physical data and transmit the data to an analysis system having a database. The system having the database, including at least prior measurements for a particular user which have been required over time, analyzes the new data received and provides for a message to be transmitted to the wireless telephone concerning the user's medical circumstances. The message can encourage the user to modify behavior in accordance with the analysis.

### SUMMARY OF THE INVENTION

In accordance with the invention, there is provided an improvement in such prior art systems. More particularly, currently there are a number of problems encountered by Caregivers with respect to serving users with chronic disease or health state. Currently, chronic disease suffers such as Type II Diabetes suffers only see their Caretaker on a quarterly basis or short consultations. This increases significantly the costs of health care. More particularly, using clinicians to make personal contact is expensive, not timely, requires multiple attempts to reach the user, is only focused on problem users because it would be prohibitive to provide feedback to all users, and the psychology of the user is not taken into consideration when providing feedback.

In accordance with the invention, personality defined feedback is provided to improve user self-care and regimen compliance. Automated feedback is provided to reduce the number of clinician personal contacts. Such feedback augments the care system and provides user contact that was previously unavailable.

In a further aspect, a system is provided which can recognize non-compliance and send appropriately designed messages to a user to coach them back into compliance. This reduces the load on the provider for human contact.

In a more specific aspect, in addition to supplying feedback to all users and taking the psychology of a user into account, the system allows for making inquires about reasons for non-compliance as soon as observed and corrective action for non-compliance can be attempted.

Like the system of U.S. Pat. No. 206/021,2316, an interface device connects to a medical device for transmitting physical data to a wireless cellular telephone. Alternatively, the medical device may be capable of transmitting the data directly over a cellular network. An example of such a device is a GSM transmission enabled meter or medical device

### BRIEF DESCRIPTION OF THE DRAWINGS

FIG. 1 is a system block diagram illustrating implementation of the invention.

FIG. 2 is a data flow diagram.

FIG. 3 is a data flow diagram illustrating use of a medical collection device employing Bluetooth.

FIG. 4 is a block diagram illustrating a backend system.

FIG. 5 is a block diagram illustrating system state/transition.

FIG. 6 is a block diagram illustrating message sets for user type.

FIG. 7 is a block diagram illustrating an initial state message set.

FIG. 8 is a detailed block diagram of the system.

FIG. 9 is a block diagram of off the shelf monitoring devices with a serial interface to a connector.

FIG. 10 illustrates a monitoring device serial interface to a connector with a Bluetooth link to a collector.

FIG. 11 illustrates a collector/server link.

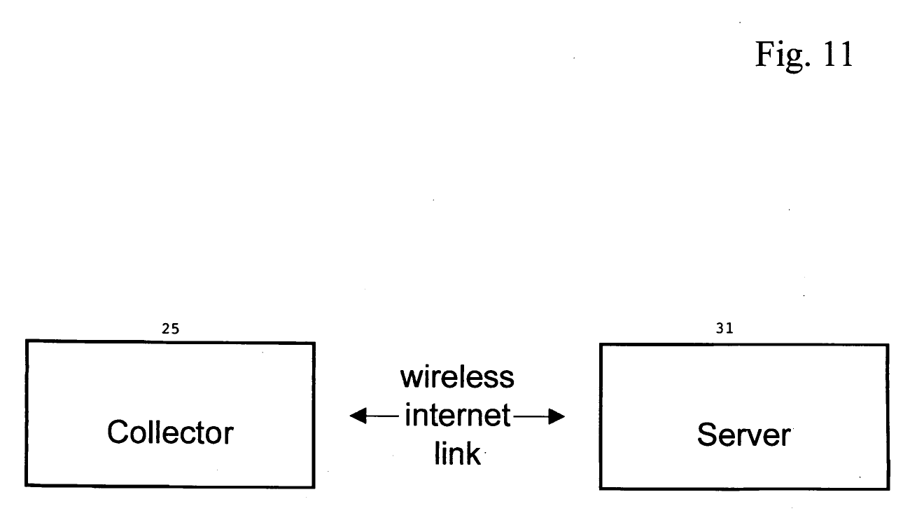

FIG. 12 is a diagram illustrating message generation processing.

FIG. 13 illustrates navigation through webpages.

### DETAILED DESCRIPTION OF THE INVENTION

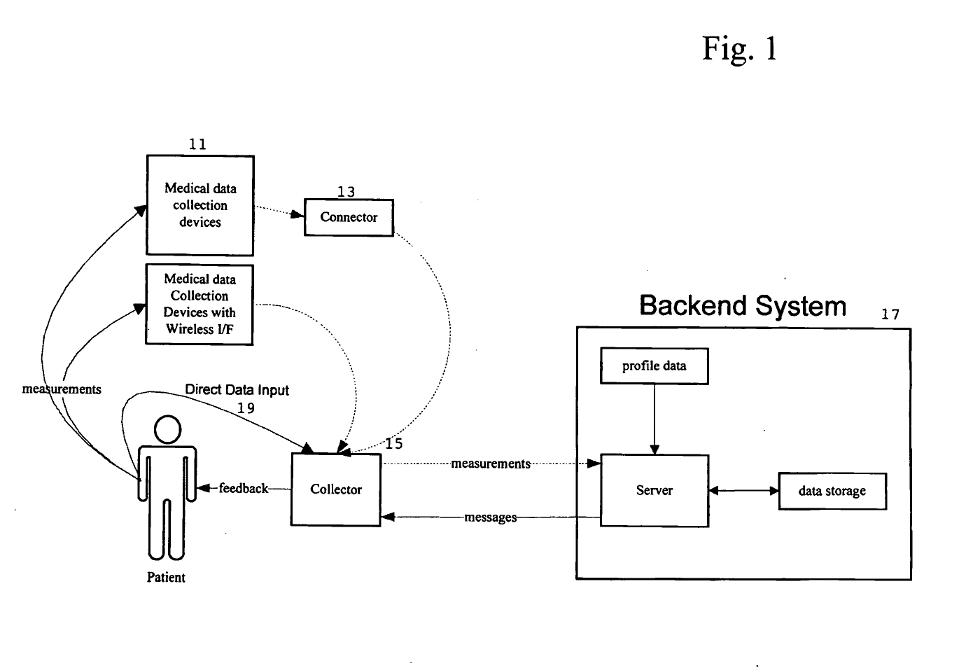

FIG. 1 is a System Block Diagram showing the flow of information and feedback from Backend Systems providing information to a patient. Also disclosed therein is a Data Flow Diagram showing in detail the various possible flows of data. A second Example Data Flow shows the use of a Medical Data Collection Device with a Bluetooth communication similar to that disclosed in said prior filed U.S. Patent Publication Number US 2006/0212316 A1.

The following is a list of Problems encountered by Caregivers with respect to serving users with a chronic disease or a health state (e.g. Obesity) that could lead to a chronic disease.

Problem list:

People with a chronic disease are often left alone to manage their disease on a daily basis. In some cases such as Type II Diabetes people only see their care taker on a quarterly basis for <15 minute consultations. This is a major artifact of the cost of health care when it comes to providing personalized service.

1. Using clinicians to make personal contact is:
a. Expensive
b. Not timely
c. Often needs multiple attempts to reach the user
d. Only focused on problem users because it would be cost prohibitive to give feed back to all users (Negative feedback system)
e. Psychology of the user is not always taken into consideration when providing feedback

There is also the Problem of “regimen compliance” from the point of view of the health care provider (payer). People that are not monitoring their disease or taking their medication end up being high cost users due to the onset of severe and expensive to treat complications.

2. Difficult to react to in a timely way to users that are not following their regimen.

3 . Medication compliance is difficult to track and reasons for non-compliance are not available in timely fashion.

14(b) Advantages over prior art

1. The prior art involves data collection only. Feedback comes from data being reviewed by the Caregiver and using personal contact to respond

2. One prior art patent application, 11/312,156 entitled Monitoring and Feedback Wireless Medical System and Method discloses an automated feedback system but does not allow for a rules based engine and personality targeted message sets.

3. U.S. Pat. No. 7,237,205 Parameter Evaluation System discloses a pre-configured device that the user would have that gave them feedback such as “Call your doctor”, Take another ______ Tablet(s)”, “Repeat measurement in 2 hours” etc. This patent is based solely on the parameters of some measurement. It does not take personality into account, coaching, regimen compliance, making dynamic adjustments. For example there is no allowance for the person not taking their medication or to help them when they experience side effects.

The invention provides numerous advantages as set forth in a nonlimiting manner below.

1. Personality defined feedback may improve user self care and regiment compliance.

2. Automated feedback will reduce the number of clinician personal contacts

3. Automated feedback will augment the care system and provide user contact that was previously unavailable.

4. The system can recognize non-compliance and send appropriately designed messages to the user to coach them back into compliance thereby reducing the load on the provider for human contact.

5. Medication side effect issues can get resolved more quickly.

6. Medication dosage adjustments can be made in a more timely way.

7. Medication abuse can be identified and reported to the provider.

8. Medication refills can be automated

The invention enables the following:

1. Supplies feedback to all users. (High Touch Positive feedback system as well as dealing with non compliant users)

2. Takes the psychology of the user into account. (The system can be pre programmed and/or learn the psychology of the individual over time.)

3. Can make inquiries about reasons for non compliance as soon as it is observed. (For example, ask questions about drug side effects)

4. Can attempt corrective action for non-compliance

5. Can compile medication statistics such as
a. User perceived medication effectiveness
b. Number of refills
c. Etc.

FIG. 1 illustrates a system block diagram including the following:

1. Medical Data Collection Device 11—This is a device that generates/collects and stores user data.

2. Connector 13—Converts wired serial data form the monitoring device into wireless data to communicate the data to an application on a mobile phone. It may or may not be needed depending on the Medical Data collection Device 11.

3. Collector 15—Application software residing for example on a mobile phone or other like device.

4. Backend System 17—Contains the Server, a database and an analysis engine that generates the feedback/message responses to the user.

5. Direct Data Input 19—The user may directly input data such as medication usage or when a device cannot electronically export its data (e.g. Some Pedometers).

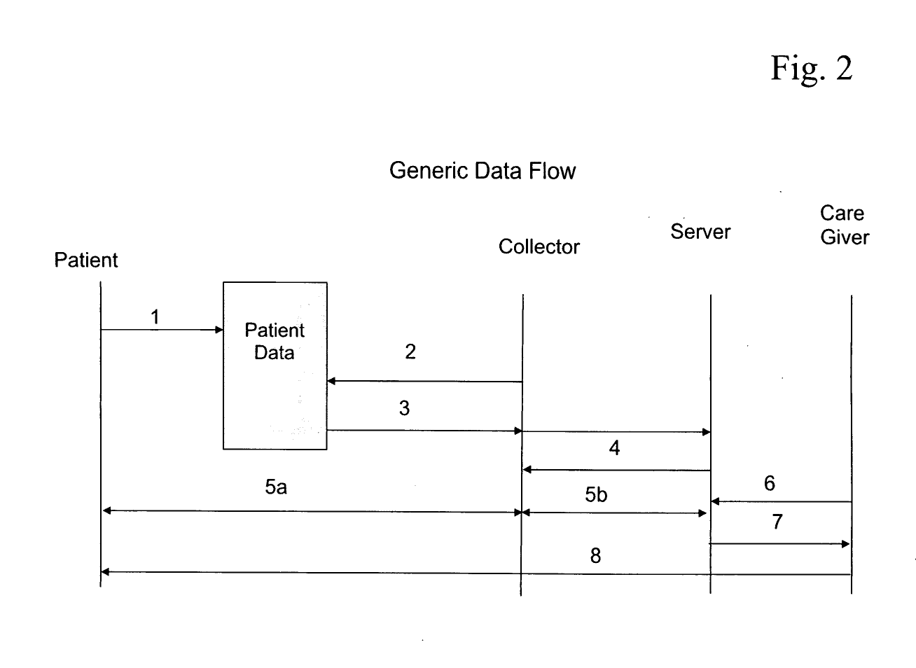

FIG. 2 illustrates a generic data flow as follows:

1. Patient has data ready to submit.

2. Patient activates application on the mobile phone to input/retrieve data

3. Data is input/retrieved by the Mobile phone and relayed to the Server

4. Server sends feedback using custom Server algorithm defined by Care Giver

5. a & b Patient sees feedback on mobile phone, Can respond to questions, Can get additional information

6. Care Giver can monitor patient data.

7. Care giver can make custom modifications

8. Care giver can also directly send feedback to the patient

It is important to appreciate that patient data can be input manually by the patient or be collected from a device that can electronically export data. In the case where the electronic export cannot interface to a mobile phone an intermediate device (Connector) is used to convert the data to Bluetooth format.

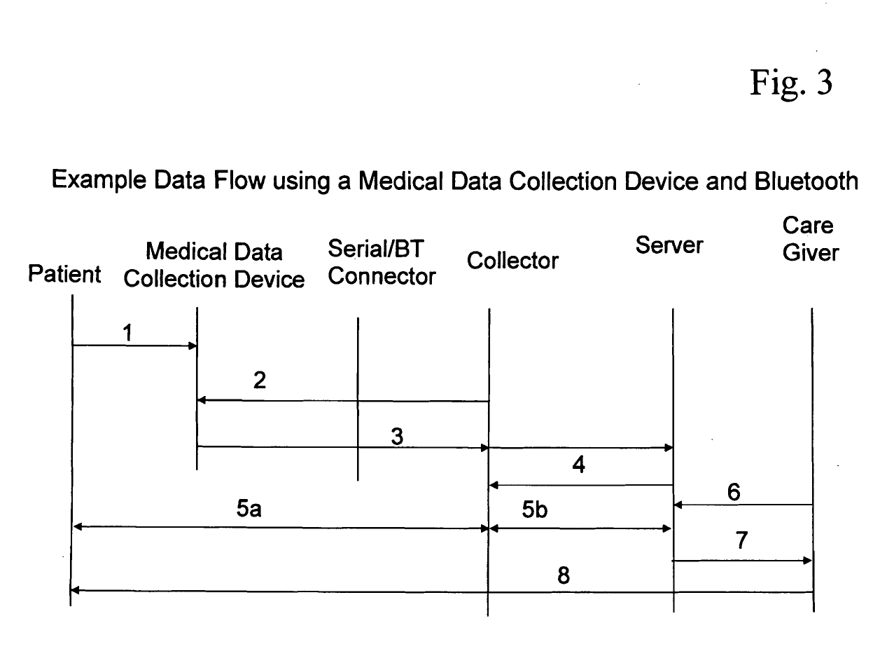

The following steps are applicable to FIG. 3

1. Patient puts data on the device

2. Patient activates application on the mobile phone to retrieve data using Serial/Bluetooth Connector

3. Data is retrieved by the Mobile phone and relayed to the Server

4. Server sends feedback using custom Server algorithm defined by Care Giver

5. a & b Patient sees feedback on mobile phone, Can respond to questions, Can get additional information

6. Care Giver can monitor patient data.

7. Care giver can make custom modifications

8. Care giver can also directly send feedback to the patient

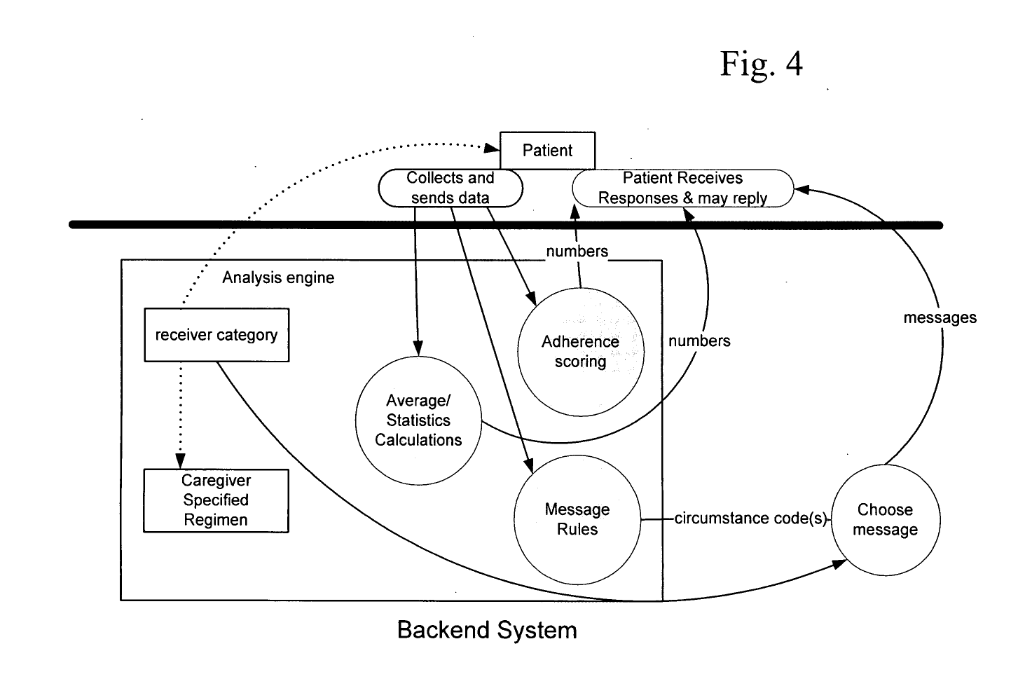

The major part of this invention is centered on the creation and use of Message Rules along with the different Message Sets from which to choose as shown in FIG. 4.

### Definition:

Receiver Category: The Receiver Category is now based on the user type, the language for the user, the condition (health problem), the psychological profile and the patient system state (explained later). In the Initial State it is chosen by default based on the Patient's data and regimens. In general it is used in the message selection process to determine which set of messages to pick from when choosing a message for a given circumstance code. Each receiver category has a set of messages corresponding to the circumstance codes defined for that condition and the reading types used to monitor that condition.

### Inputs for Message Rules:

The Message Rules are based on four types of inputs:

1. Psychological Profiling

2. User Regimens

3. Drug/medication Information

4. Run Time Change of State

Before going into how the inputs create message rules, it's best to first define message sets.

### Message Sets:

The system contains multiple message sets each corresponding to different relations resulting from the inputs for message rules. It is important to appreciate that patient data can be input manually by the patient into the mobile phone, can be collected by the mobile phone from a device that can electronically export data, or can be sent directly to the server by the device using the cellular network. In the case where the electronic export cannot directly interface to a mobile phone, an intermediate device (connector 13) is used to connect the device to the mobile phone using Bluetooth.

A sample set of message sets are given as follows:

1. Psychological profiling leads to putting users into different types of behavior classes so a set of messages will exist for each behavior class. The messages are optimized by health care professionals and medical experts to get the best positive response from the user. Within a psychological profile there are sub categories of messages depending on:
a. User Regimen
b. Medication Information
c. Run time change of user state

### User Regimen:

The user regiment consists of variety of different elements and can incorporate multiple disease/co-morbidity states for each user. In the initial system there are the following items:

1. Frequency of reporting. (e.g. 2 reports per day)

2. Frequency of reminders from the system (e.g. 1 reminder at 5:00 PM per day if no data reported)

3. Auto start up of the application at a specific time.

4. Measurement data targets

5. Medication compliance

6. Lifestyle compliance (Exercise and diet responses)

7. Notifications (e.g. The system can contact a 3rd party when a pre specified event occurs)

### Medication Information:

Medication or drug information can include but is not limited to:

1. Dosage

2. Time of day

3. Side effects

4. Refill amounts

5. Refill supplier

6. Run time counts

### User States:

In the initial system, there are four user states along with transition conditions between states. They are:

1. Initial State

2. Reporting Data on Regimen

3. Reporting Data off Regimen

4. Not Reporting Sufficient Data

Initial State: All new users start in Initial State. This is where Psychological profiling occurs. There is some amount of coaching and a generic message set is used. Time spent in Initial State is set by the caretaker. After the time period is over, the user moves to one of the three other states depending on their performance. Only the caretaker can transition a user back into Initial State.

Reporting Data on Regimen: This is based on percentage compliance and percentage of on target data set by the caretaker.

Reporting Data not on Regimen: The user is meeting the percentage compliance set by the caretaker, however the user is not meeting the percentage of on target data.

Not Reporting Sufficient Data: The user is not meeting the percentage compliance set by the caretaker.

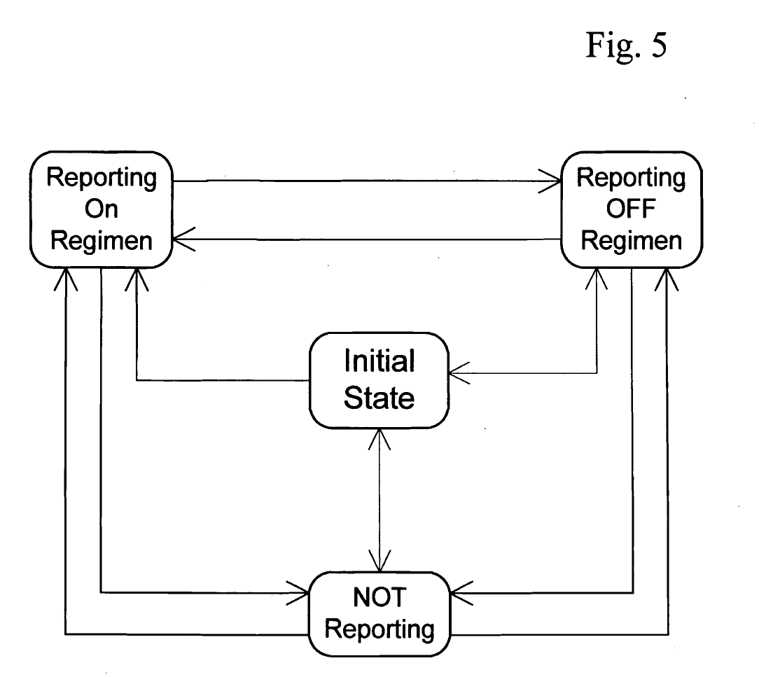

FIG. 5 illustrates a state/transition diagram. Except possibly for the Initial State, each of the States (Reporting ON Regimen, Reporting OFF Regimen, NOT Reporting) could have assigned to them individual message sets based on user characteristics as illustrated below.

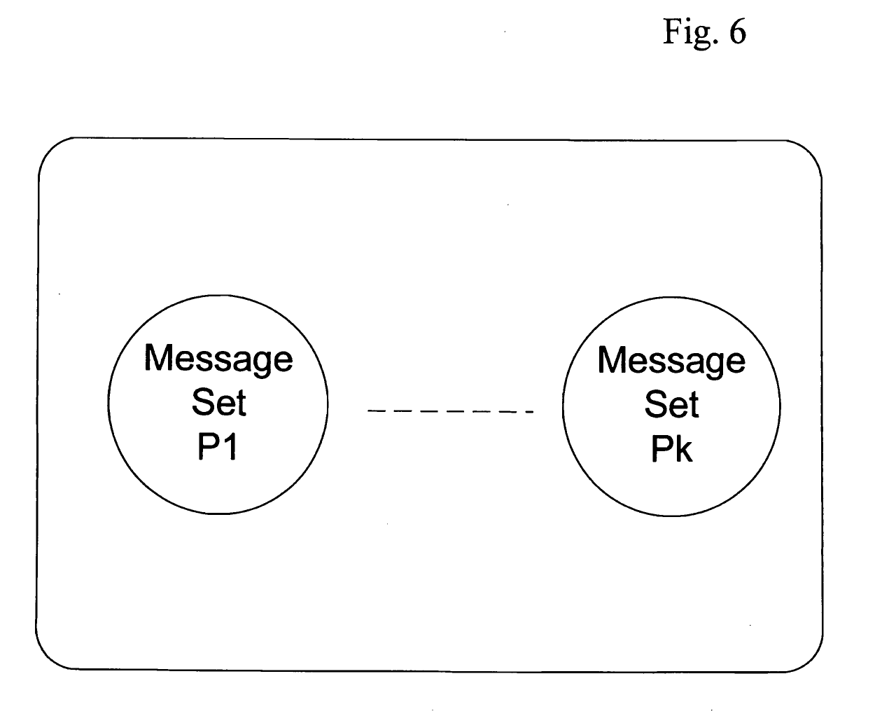

FIG. 6 represents k-message sets for User Types P1 through Pk and states Reporting ON Regimen, Reporting OFF Regimen, and NOT reporting with Initial State as optional. The message sets will be different depending on the state but will be consistent with the User Type across states.

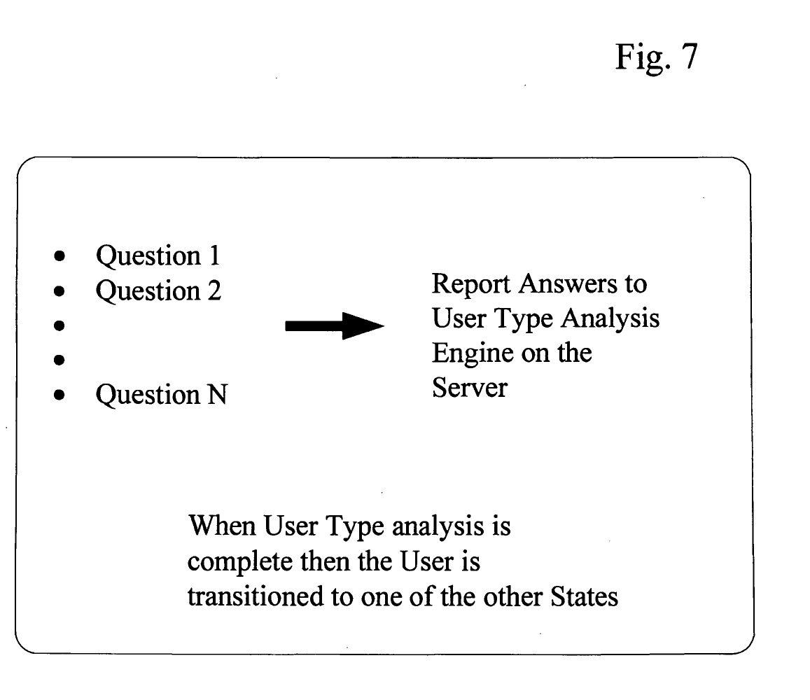

The Initial State as shown in FIG. 7 can also have message sets based on User Characteristics but it can also be used to determine the User Characteristics. This would be done by presenting a series of questions to the users as they grow accustomed to the system and then channel the user into the other states targeting the appropriate message set.

Having broadly described the invention, the following provides more specific detail about implementation.

The following is a list of the actors in the system, i.e., the entities that are visible to a user of the system:

Patient (or User)—User of the system; our targeted user has a chronic medical condition, such as diabetes, that can be alleviated by changing lifestyle habits.

Monitoring Device—Any of the off-the-shelf devices that measure one or more individual health parameters: blood pressure cuff, glucometer, etc. To be useful in the system, the device must provide some way of communicating the measurements taken with it to another device.

Connector (Bluetooth converter)—Device for obtaining data from a monitoring device and transmitting it wirelessly via the Bluetooth communication protocol.

Collector—A program that runs on a mobile telephone with Bluetooth capability for communication with Bluetooth converters, and with internet data capability for communication with the Server.

Server—Collects medical measurement data coming from the Collector; generates and sends messages to users, guardians, and clinicians, as well as, providing a web interface for account configuration and data access.

Guardian—Guardian of one or more minor users; a patient's profile may indicate that messages are to be sent to his guardian under certain conditions.

Clinician—Health care provider for the user. The clinician sets some of the parameters used to choose what messages to send to a patient. A clinician can also select an option to receive patient event notifications.

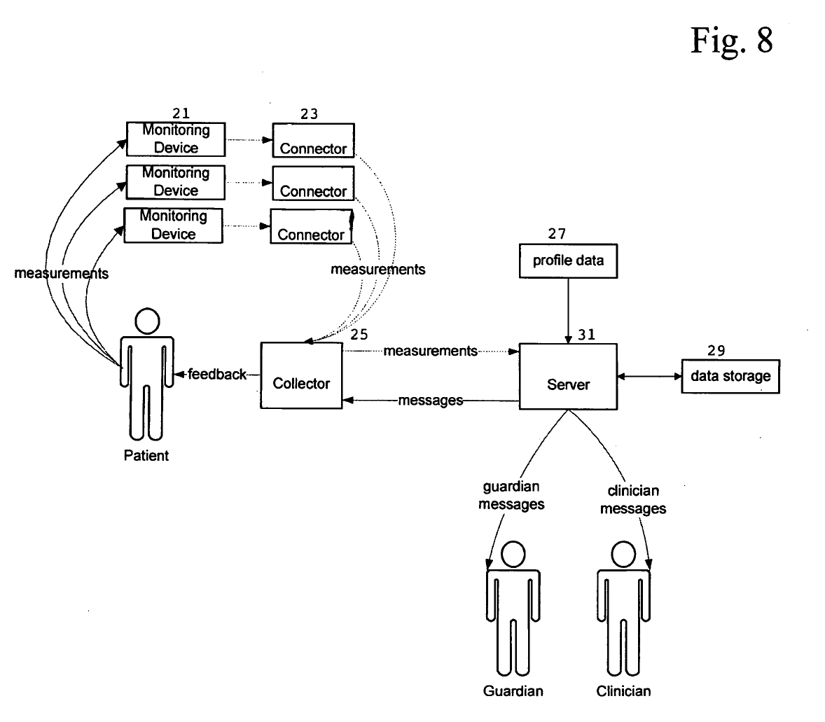

In greater detail than FIG. 1, fundamental parts of the System and the flow of data are diagrammed below in FIG. 8:

The following explains each item:

Monitoring Devices 21 allow the patient to take measurements of different reading types and collect that data with the Collector.

Connectors 23 are Bluetooth converters allowing the monitoring devices and the Collector 25 to communicate with each other.

Profile Data 27 contains information about each user: which medical parameters are being measured; parametric data such as birth date, sex, height, and weight, and data that help make decisions about what messages to deliver. The text of messages can be different for adults, teens and juveniles, men and women, etc.

Data Storage 29 contains the data collected from the monitoring devices and all other information needed about users, regimens, etc.

The following is a more detailed description of collector 25. A patient takes one or more medical readings using off-the-shelf monitoring devices. He might take these readings regularly several times a day. Under the user's control, the collector 25 collects these measurements and delivers them to the server 31.

To collect data, a collector program uses Bluetooth wireless communication technology to send commands to monitoring devices and receive data in response to the commands via the connector.

The server 31 operates as follows: A patient's clinician sets the ranges of acceptable values for measurements and the number of times per day or week they are to be taken. The server 31 compares the measurement values and times taken to this “regimen” specified by the clinician for the patient. Server software uses the results to generate and send feedback messages to users and, if appropriate, to guardians and clinicians.

Feedback can be different for different categories of patient, so that the intended message can be delivered in the most effective way for that user. The goal of the feedback is to encourage better self-management of the condition by keeping the patient informed with timely, accurate information.

The server 31 software accepts data from collectors 25 and returns or sends messages based on the data. One server 31 installation supports many collectors 25, each configured for a specific user and his monitoring devices. All these users might suffer the same or different chronic conditions.

All transmission, storage, and access of patient identifiable information (i.e., Protected Health Information) collected and/or stored by the System conforms to US HIPAA Security and Privacy regulations.

All communications between the collector software and the server 31 are encrypted to protect the privacy of the data.

The configuration of a kit for a patient begins with the creation of the patient account on the website. The Clinician creates the patient account selecting the patient's condition, the devices the patient will use, the regimen for each device, etc. Additionally, the clinician selects the type of phone the patient will use and enters the patient's cell phone number. After the patient account is created, the Server sends a message to the patient's phone which contains the URL needed to download the collector application. The patient is then able to select the URL in their message reading program on the phone and download a customized version of the Collector application.

The installation process is fairly simple, but the patient will need to answer a few questions about whether to install the application, where to save it, etc. On first running the application the patient will be guided through the configuration process. If the clinician specified the type of medical device the patient has and/or the Bluetooth ID of their Connector device the configuration process is simplified.

If the clinician did not specify the type of medical device the patient is using then the patient is prompted to select the medical device from a list.

If the clinician did not specify the Bluetooth ID for the patient then a Bluetooth search is done to find available devices. The collector 25 first looks for a connector device 23 with the friendly name matching the device type. If none is found or if more than one is found, the patient must choose from a list of Bluetooth IDs to select which one is theirs.

Under normal circumstances the patient will not need to answer any questions during the configuration process.

During the initial configuration process on the collector 25 a check is made to verify that the downloaded version of the collector application is the correct version for the phone it is running on.

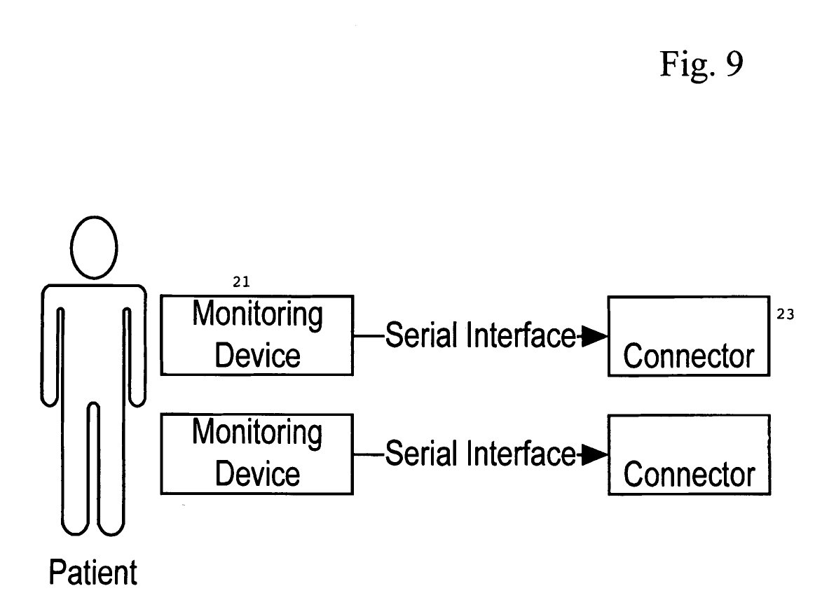

As shown in FIG. 9 the system works with of-the-shelf monitoring devices 21 that are capable of delivering their measurements electronically. Typically the device has a serial interface, and is plugged into a connector 23 (Bluetooth converter) device with a manufacturer supplied communication cable.

As shown, one user can have multiple monitoring devices, with one connector (Bluetooth converter) for each. Each monitoring device may collect one or more measurements from the user. Examples of the Bluetooth connector are disclosed in patent application Ser. No. 11/312,156.

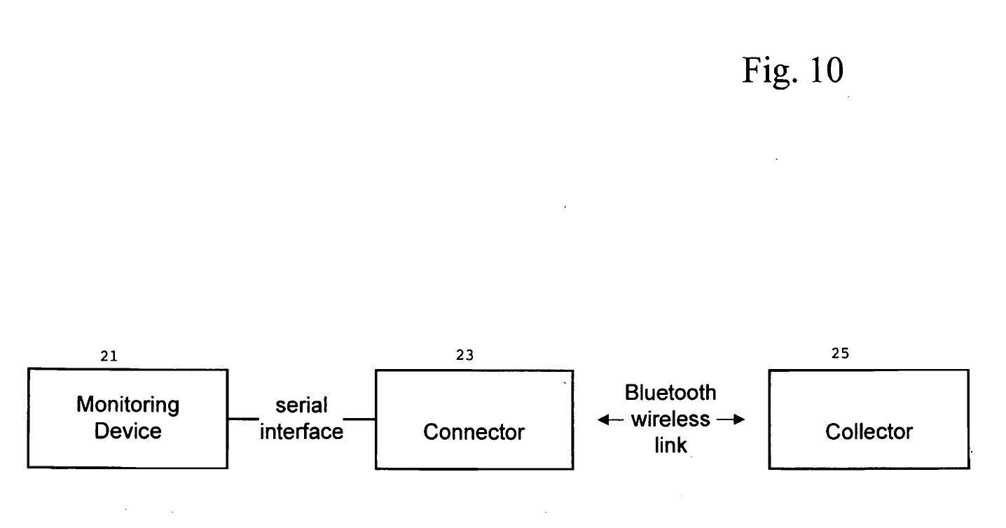

As shown in FIG. 10 the connector 23 is a device that communicates via serial cable to a monitoring device 21 and also via Bluetooth communication to the collector 25. The connector 23 enables communication between the collector 25 and a monitoring device with a serial interface.

The connector 23 uses the 10 meter Bluetooth communication protocol and so as the required range of up to 30 feet (10 meters).

The connector 23 does not interfere with normal operation of the monitoring device, and it is small enough not to interfere with the monitoring devices portability.

Communication with a monitoring device 21 is initiated by the user operating the collector 25, which in turn controls the Bluetooth communication with the monitoring device 21.

When a connector 23 is first plugged into a monitoring device 21, it needs to be configured for use with that monitoring device 21 using the collector 25. This takes place during kit configuration.

Before a collector 25 is first used it must be configured for the specific monitoring devices 21 and connectors 23 it will use. This is done during kit configuration.

There are two kinds of monitoring devices supported by the collector 25:

1. Stores measurements as the user takes them. At a later time, the user may use the collector 25 to obtain the measurement values and the times that they were taken.

2. Does not store measurements, but instead sends the measurement values out through its communication port at the same time the user is taking the measurement. To have the collector 25 obtain data from such a device, the user needs to operate the collector 25 so it is ready to read the data as it comes in.

The collector 25 stores the necessary software for each model of supported device. When the user selects the collector option to collect and send data, the collector 25 communicates with each device to either retrieve its data or to have the device operate a data measurement so the collector 25 may receive data while the measurement proceeds. In the latter case, the collector 25 also displays instructions to the user on what things to do and in what order to operate the device properly for data collection.

Once the collector 25 has collected data from all available monitoring devices, it sends all the data to the Server for analysis and storage.

In this version of the system, the collector 25 may be one of the following: Nokia 6620 and Nokia 6682 mobile phone running Java MIDP 2.0 and capable of Bluetooth communication using the Java API for Bluetooth, JSR 82 and internet enabled using GPRS on the Cingular network. Using this collector 25 device, no personal computer is necessary to operate in the system. Mobile phones are normally easy to carry, making it easy for a user to travel with the system.

The Collector UI (user interface) includes the capability to:

1. Enter basic patient data. Specified in 0, “Configuration Settings.”

2. Collect and upload monitoring device data to the Server. The procedure is described in 0, “7.3. Collecting and Sending Data.”

3. Receive and display messages from the Server. Described in 0, “.”

The collector 25 provides support for internationalization (language specific versions).

A collector application will make use of any software data verification functions that a supported monitoring device provides in order to verify the integrity of the data transmitted.

A collector main screen displays the last data submission date, current adherence score, and current average reading.

Collector software can be built to only include the communication software for a specified subset of supported monitoring devices. This allows delivery of Collector software with support for any combination of monitoring devices that the system is capable of supporting.

Interruptions of Collector Operations of Collector Operations.

Since the collector 25 runs on a mobile telephone, it is possible that some event (such as an incoming phone call) will interrupt any collector 25 operation being performed by the user. The collector program handles interruptions by allowing an operation to proceed until it requires user interaction. At this point the operation is suspended until the call interruption is finished. This is inline with the requirements of mobile phone manufacturers and carriers for handling call interruption. There is no opportunity for data loss or for the user to have to restart data collection.

### Configuration Settings:

Each collector 25 must have the patient's username and password to communicate with the Server 31. During initial configuration the username is entered and cannot be changed. The collector 25 provides a way for the patient to change their password after configuration is complete.

Note that changing the password on the collector 25 does not change the patient's password on the server. In order to change the password accepted by the server the patient or server administrator must use the server interface. Best practice would be to change the password on the server 31 first and then change the password on the collector 25 to match.

The user is also allowed to change the default font size (small, medium, large) used throughout the application.

Collecting and Sending Data.

The collector 25 provides an option that causes it to collect measurement data from all monitoring 31 devices configured for collection and send the data to the server 31. In case a set of previously collected data was unable to be transmitted to the server 31, it also provides an option to send that data to the server without performing another collection.

To collect measurement data and send it:

1. The patient selects an option on the collector 25 to start collecting and sending data.

2. The collector 25 validates that the monitoring device is set with the correct unit of measure UOM). If the UOM is incorrect a message is displayed to the patient and the Collector 25 attempts to set the UOM to the correct setting if it is supported by the monitoring device. If not supported, an alert is displayed requesting the patient change the setting on the device.

3. The collector 25 validates that the monitoring device 21 is set with the same time and date as the phone. If the time and date are not the same a message is displayed to the patient and the collector 25 sets the correct time and date on the device if that function is supported by the monitoring device. If not supported, an alert is displayed requesting the patient change the time on the device.

4. The collector 25 attempts to collect data from each of the configured devices.

5. While collecting data, the collector 25 displays a screen to the patient indicating that collection is in progress. It also provides an option for the patient to halt the process.

6. Any readings returned by the monitoring device 21 that are marked as control readings, that are marked as invalid readings, or that have a time that is more than 2 hours in the future or 28 days in the past are discarded. A message is displayed to the user informing them that readings were discarded.

7. If collection of measurements from any of the devices was successful, and if data erasure is supported via communication link by the monitoring device, the collector 25 sends a command to the monitoring device to erase the measurements stored on the monitoring device.

If the patient has collected data as described above, the steps below are taken automatically. If the collector 25 cannot send the collected data to the server 31, it stores it. If the collector 25 has stored data and the user selects the option to send it to the server 31, it also follows the steps below.

Also, if the patient collects data that cannot be sent to the server 31, and later performs another data collection operation, the collector 25 sends both the stored and the newly collected data to the server 31 automatically.

1. The collector 25 sends the collected measurement data to the Server 31 using encrypted communication to protect the privacy of the data. During the send process the collector 25 displays the reading values being submitted for the user to view.

2. The collector 25 receives feedback messages for the user after completing the send operation. It displays either:
a. Any feedback message(s) it gets from the server,
b. A message indicating that the send was successful, but that feedback messages are not yet available, or
c. An error message indicating that the send may not have succeeded, and any information it has about why. This message is written for a non-technical user, though it may contain information for reporting to technical people.

3. If the collector 25 obtains data from the monitoring devices but then cannot send it to the server 31, it stores the collected data so it may send it at a later time. If the collector 25 receives an acknowledgement from the server 21 after sending in data, it discards that data from storage on the collector 25.

### Response Message to Data Submission:

Messages are targeted at helping the patient manage their condition. They are tailored to the specific patient by the medical parameters entered by his clinician, and to the patient's specific situation by comparing the data sent from his monitoring devices to those parameters.

Messages to the patient from the server 31 normally arrive in response to the command to collect and send data. This is the default and desirable way to receive messages: immediately, without requiring further action on the part of the patient. These messages do not arrive by email or short message service; they are part of the data exchanged between the collector 25 and the server 21 at the time of data send.

Messages from the server 31 to the guardian or clinician, as well as to the patient, can be sent as an email to an address configured for the recipient (note that many mobile phones take email, so the email may be delivered to a phone with this feature or to a more traditional email client).

Each patient's profile specifies the clinician and/or guardian to which messages are sent. Each clinician and/or guardian has their own profile containing their message delivery preferences and address.

The server 31 response is presented to the user with the following three screens. The first two screens contain only one option for the user “Next”. The final screen will have a “Done” command.

### Graphical Response Screen:

A screen displays a graphical representation of the patient's current status. Two arrows display the movement of the patient's two-week average adherence score and two-week average glucose reading value when compared to the current data. An arrow pointing up and right indicates the current data has increased from the 14-day average value, an arrow pointing down and right indicates the current value has decreased from the 14-day average value, and an arrow pointing straight across indicates the current value and the 14-day average value are the same.

Each arrow is colored red if it is a bad direction, green if it is a good direction, yellow if it is the same or no color if there is no notable good or bad change (applies only to glucose average arrow). For the Adherence arrow, if both values are 100, then the arrow is green.

The current value is calculated for the current time period, which is defined as the time from the last submission until the current submission. For multiple-readings-per-day regimens the required number of readings is translated to 1 reading per x number of hours by just dividing it out, so that a 4 times a day regimen requires 1 reading every 6 hours. Therefore if the last submission was between 7 and 11 hours ago, only one reading is required by their regimen.

There are some special cases that apply to the color of the glucose average arrow, therefore specifically stating what color the arrow is in all possible cases is necessary:

If the 14-day average is on target and the current average is on target then the arrow points straight across and is green.

If the 14-day average is below the target low value then the arrow points in the direction of change, but has no color

If the 14-day average and the new readings average are the same and are not in the target range then the arrow points straight across and is yellow.

If any new reading is below the target low and the arrow points down then the arrow has no color but points in the direction of change.

In all other cases the arrow points in the direction of change and the color is green if the current is less than the 14-day average or red if it is greater.

Listed beside the arrow are the current value and the 14-day average value as shown on a typical screen on a collector 25.

### Text Message Response Screen:

Any text message received from the server is displayed here using the font size set by the patient in the program settings.

### History Options Screen:

Finally a list of options is displayed that the user can select to view more information. The options are the same as specified in the Viewing Historical Data section.

### Unsolicited Messages:

The server is capable of sending the patient messages that are not in response to submitted readings. These messages are usually focused around reminding the patient to take and/or submit their readings. These messages arrive on the patient's phone as an SMS message. When supported by the phone the collector program will register for and receive incoming SMS messages sent from the server and display these messages directly in the application rather than the user reading these messages in their SMS inbox. Additionally the collector will auto-start when an incoming SMS is received from the Server.

### Viewing Historical Data:

The History Page provides a list of options the user can select to see information about past readings, scores, averages, and messages. The following defines each option and what is displayed when that option is selected.

### Readings:

The last 14 days of submitted readings are displayed in reverse chronological order.

### Readings Graph:

A scatter plot graph of the previous 7 days or 14 days of readings with glucose value on the y-axis and time on the x-axis is displayed.

### Average Glucose Value Graph:

This screen displays a scatter graph of the user's average glucose reading value for each week for the past 6 weeks. This is calculated as a rolling average that is updated each time they submit data. So the last value on the chart is the current week, which starts with the current day and goes back six more days. The prior value on the chart is the 7 days prior to that.

### Average Adherence Score Graph:

This screen displays a scatter graph of the user's average adherence score value for each week for the past 6 weeks.

### Messages:

Upon selecting this option the user sees the last message that was sent to them as a response to submitting data. There are next and done command options that allow the user to see the next previous message or to return the history list page.

### Monitoring Devices:

The following monitoring devices are supported in the system:

Abbott Therasense Freestyle Flash Glucometer.

Abbott Therasense Freestyle Glucometer.

Bayer Ascensia Contour Glucometer.

Bayer Ascensia Breeze Glucometer.

Bayer Ascensia Elite XL Glucometer.

Bayer Ascensia Dex2 Glucometer.

Johnson & Johnson Lifescan OneTouch Ultra Glucometer.

Johnson & Johnson Lifescan OneTouch Ultra2 Glucometer.

A&D Medical UA-767PC Blood Pressure Meter.

A&D Medical LifeSource UC-321P Scale.

A&D Medical UA-767PBT Blood Pressure Meter.

A&D Medical LifeSource UC-321PBT Scale.

### Data Collection Process and Exceptions:

In general the Collector 25 works with medical devices 21 by performing the following steps:

1. Get the time from the meter.

2. If the time was not correct, set it.

3. Get the unit of measure from the meter.

4. If the unit of measure was not correct, set it.

5. Retrieve only the new readings from the device.

Below are deviations from the above procedure:

None of the Bayer glucometers, the One Touch Ultra, or the A&D BP meter supports a workable method for retrieving only the latest readings; therefore the Collector 25 invokes the clear command after retrieving the readings from the device.

The LifeSource UC-321P scale does not use time, nor does it support changing the unit of measure via the communication protocol. It does allow the user to change the uom via a switch on the device. If the unit of measure is not set to LB during data collection and error is displayed and the user is prompted to change the uom to LB and retake the reading.

The OneTouch Ultra and Ultra2, Freestyle and Freestyle Flash meters only support mg/dL units of measure via the communication protocol, so there is no need to check or change it.

The A&D Medical PBT devices support time but the Collector does not use it as the readings are collected in real time, so there is no need to set the time. For uom the comment for the UC-321P applies here as well.

### Unit of Measure:

The System is standardized to use only one unit of measure per device type. The following table lists the units of measure:

| | |
|---|---|
| glucose | mg/dL |
| blood pressure | mmHg |
| weight | LB |

### Server:

The server 31 runs on Debian GNU/Linux 3.1 and will support a minimum of 500 simultaneous users.

The system helps patients with chronic medical conditions by examining data collected from biometric monitoring devices and returning messages to help them better manage their condition. This section specifies Server's translation of the data it receives into messages.

The system separates the choice of a message into two steps: analysis of the patient's data and choosing the message to send.

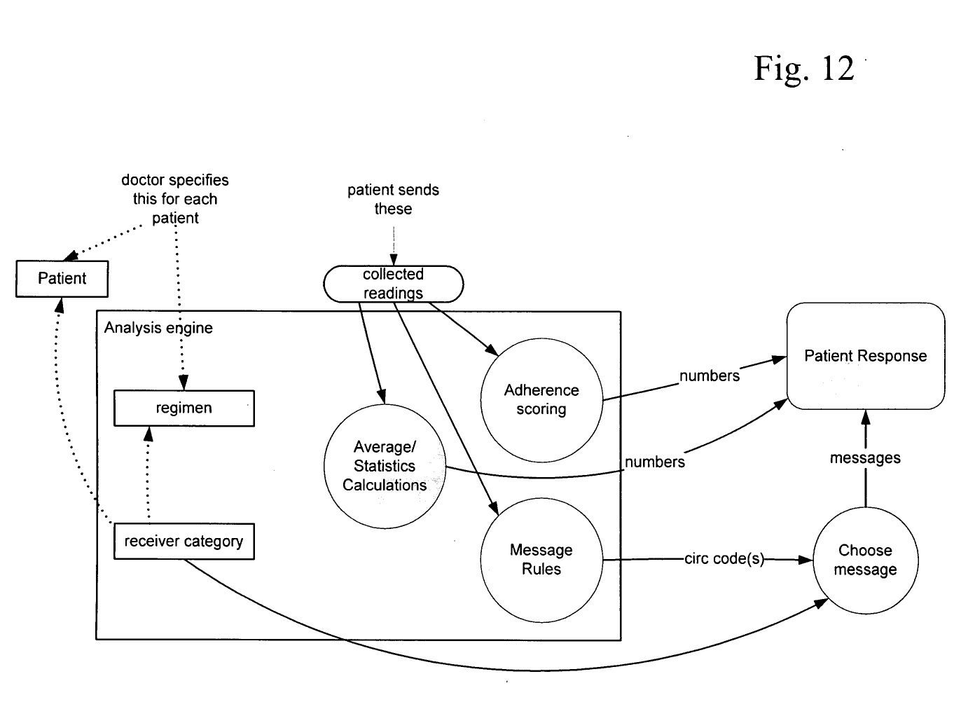

The following diagram of FIG. 12 shows an overview of message generation processing.

### Message Generation Overview:

The diagram of FIG. 12 shows that the clinician creates the patient and their regimen(s). When the server 31 receives readings from the patient, it calculates the adherence score based on the regimen, calculates the average glucose values, and then runs through the rules defined for each condition-reading type pair; this determines the patient's circumstance codes. From the circumstance codes generated the next step is to select which circumstance codes will be used to generate the patient's message. The receiver category is then used to pick the actual text message.

### Regimen:

A regimen defines the attributes of a given reading type for a patient. A patient may have multiple regimens. The attributes set in a regimen vary with reading type.

All Regimens have the following attributes:

Condition

Reading type

the period over which the patient is to take a number of readings, either daily or weekly

How many readings the patient is to take over the period, from 1 to 10

Number of missed readings in a week that should result in a notification to the clinician.

Yes/no for whether to notify the clinician of abnormal circumstances as defined in the analyzer.

Glucose regimens have the following additional attributes:

Target low and high which define the target range

High and Low which define the midway ranges

Danger low and high which define the danger thresholds

Blood Pressure and weight do not have value analysis, they are only analyzed for adherence to the regimen.

### Receiver Category:

The Receiver Category is based on the user type, the language for the user, and the condition. It is chosen by default based on the Patient's data and regimens. It is used in the message selection process to determine which set of messages to pick from when choosing a message for a given circumstance code. Each receiver category has a set of messages corresponding to the circumstance codes defined for that condition and the reading types used to monitor that condition.

### Data Analysis:

The following section details the analysis done by the Server every time the patient submits new readings. There are three parts to the data analysis: calculate adherence scores, calculate average values, and run through all rules available for the patient's condition. Each part can generate zero or more circumstance codes that are then used during the message selection process.

### Adherence Scoring:

An adherence score is an evaluation of a patient having taken the required number of measurements as defined in their regimen. It is expressed in integers as a percentage of readings taken to the number required. The score is calculated using data from the last 14 days. As the first day the patient is on the system may be a partial day, the first day is excluded from scoring. The score is a percentage and thus cannot go above 100 or below 0.

The actual calculation of the adherence score involves calculating a score for each day and then averaging those values. This removes the possibility of the patient submitting more than required one day and fewer than required the next and getting a high adherence score.

As part of the response message to the Collector the patient's adherence score (14 day average) is returned along with their current score. The current score is the percentage of readings taken to the number required over the time period from the last submission to the current submission. For instance, if a user is to take one reading per day and they miss a day, then submit one reading 48 hours after their last submission their current score would be 50%. A four per day regimen that submits one reading at a time, the current score would be based on requiring one reading every six hours.

In addition to the current and 14-day average score, the Server also returns the average score for each of the last six, seven-day periods. This information is used to provide the user a graph of their average score by week.

### Average Reading Value:

During the analysis process the Server calculates numerous average values based on the glucose readings the patient has submitted. The current average value, defined as the average of all of the readings the user has just submitted, is calculated in returned. Additionally, the 14-day average is calculated by averaging all readings submitted for the last 14 days.

In addition to the current and 14-day average value, the Server also returns the average value for each of the last six, seven-day periods. This information is used to provide the user a graph of their average value by week.

### Priority Rules:

In the rules section to follow, each rule results in a circumstance code. Each circumstance code is assigned a priority rule which determines how that circumstance code is evaluated when determining which circumstance code to use in message generation for a patient. The following priority rules exist:

1. Override Priority—override priority means that the circumstance code is sent every time it is generated and only other override priority codes could be sent with it. Meaning that it overrides any LRS priority codes.

2. Least Recently Seen (LRS)—LRS priority means that the circumstance code that was least recently seen by the patient is the one that should be sent this time. This is the default priority and as long as there are no override priorities one LRS code will be sent (assuming one was generated).

3. Always Send—always send priority means that this code gets sent as an extra in addition to sending the LRS code.

### Glucose Value Rules.

The following section details the rules used for analysis of glucose values for type 1 and type 2 diabetes. Each section defines the rule, specifies its application, lists the resulting circumstance codes, and lists any parameters passed with the circumstance code. The application specification section states whether the rule applies to type 1, type 2, or both and any other restrictions, such as the rule may only apply to regimens of 2 or more times per day.

### Trend in Average Value.

This rule uses least squares analysis to find a significant upward or downward trend in the glucose values over a given time period The function is run for three time periods: the last 7 days, the last 14 days and the last 30 days. A significant trend is measured using the slope of the resulting function. Upward trends must be above the target range. For downward trends there are two circumstance codes, one for trends that are below the target range and one for trends above. Below the target range is defined as the reading values from the last 1/7 of the time period average less than the target value.

Application: type 1 and type 2

Circumstance codes: valtrdupp, valtrddwn, valtrddwnlow

Parameters: timePeriod (last week, last two weeks, last month)

Priority: Least Recently Seen

### Messages:

valavgtrdupp—Your overall glucose average is rising over the past ${timePeriod}.

valavgtrddwn—Great Job ${nick}! Your glucose average over the past ${timeperiod} is trending down. Keep it up.

valavgtrddwnlow—Your glucose average over the past ${timeperiod} is trending down. Try to keep it in the target range.

### Trend at Specific Time of Day.

This rule uses least squares analysis to find a significant upward or downward trend in readings taken at roughly the same time of different days. The function is run for three time periods: 7 days, 14 days, and 30 days. It breaks each day down to 5 time periods to analysis as defined by the timeOfDay parameter.

Application: type 1 and type 2 but only applies to regimens of more than one reading per day.

Circumstance codes: todtrdupp,todtrddwn, todtrddwnlow Parameters: timePeriod (last week, last two weeks, last month), timeOfDay 5 am-10 am (morning), 10 am-2 pm (mid-day), 2 pm-5 pm (afternoon), 5 pm-9 pm (evening), and 9 pm-5 am (nighttime).

Priority: Least Recently Seen

### Messages:

todtrdupp—Your ${timeOfDay} readings seem to be trending up over the past ${timePeriod}. Make sure you keep an eye on them.

todtrdupp—${nick}, your ${timeOfDay} readings are trending up over the past ${timePeriod}. Please make sure you understand the implications of this.

todtrddwn—Your ${timeOfDay} readings are trending down over the past ${timePeriod}. Great Job!

todtrddwnlow—Your ${timeOfDay} readings seem to be trending down below the target range over the past ${timeperiod}. Keep working at staying within your target range.

### On Target Reading(s).

This rule requires that the most recent reading submitted is on target reading and that the patient has a current average above the target zone and that there previous submission did not have any on target readings and there are no danger high or low readings in the current submission.

Another portion of this rule applies if the returned 14-day average glucose value is within the target range and it was not in the target range for the previous two submissions and the patient has been on the system for at least 14 days.

Application: type 1 and type 2

Circumstance codes: onntgt and onntgtavg

Parameters: none

Priority: Least Recently Seen

### Messages:

onntgt—Good work ${nick}. Your last glucose reading was on target. Keep it up.

onntgtavg—Great work ${nick} on getting your 14-day glucose average within your target range!

onntgtavg—Your glucose average for the past two weeks is in the target range, great job!

### Three Low Readings at Same Time of Day.

This rule looks for three readings below the target low value set in the patient's regimen on three consecutive days. Each reading must be within one hour of the readings before and after it. The third reading in the group must be a newly submitted reading.

Application: type 1 and type 2 but only applies to regimens of more than one reading per day.

Circumstance codes: conlow

Parameters: timeOfDay

Priority: Least Recently Seen

### Messages:

conlow—Are you aware that you have had low ${timeOfDay} readings for 3 days in a row? Do you know what action to take?

conlow—That's three low ${timeOfDay} readings in a row ${nick}. Is there something you can do to address this?

Three Very High Readings at Same Time of Day.

This rule looks for three readings above the very high value set in the patient's regimen on three consecutive days. Each reading must be within one hour of the readings before and after it. The third reading in the group must be a newly submitted reading.

Application: type 1 and type 2 but only applies to regimens of more than one reading per day.

Circumstance codes: conhgh

Parameters: timeOfDay

Priority: Least Recently Seen

### Messages:

conhgh—You've had ${timeOfDay} readings over your target range 3 days in a row. Are you aware of what you can do bring them back into target?

conhgh—Take notice of your ${timeOfDay} readings. They've been above target the last few days. Do you know what actions to take?

Average Glucose Numbers.

This rule takes care of calculating the average glucose numbers that the Collector needs for displaying the graphical response screen and the average glucose graph. The 14-day glucose average is calculated for the previous 14 days from the current time and day. The current average is calculated for all readings submitted from the last submission to the current submission. The weekly averages are calculated as the average from the current day back seven days and then continuing back another seven days and another, etc. for a total of 6 weeks.

Application: type 1 and type 2

Circumstance codes: valavg, valwek

Parameters: val, curavg, 14davg, arwcol, arwdir

Priority: Always Send

Glucose Adherence Rules.

### Trend in Adherence Score.

This rule uses least squares analysis to find a significant upward or downward trend in the rolling 14-day adherence score. The function is run for three data sets: the last 7 days, the last 14 days and the last 30 days. A significant trend is measured using the slope of the resulting function.

Application: type 1 and type 2

Circumstance codes: adhavgtrdupp and adhavgtrddwn

Parameters: timePeriod

Priority: Least Recently Seen

### Messages:

adhavgtrdupp—Your adherence score has been on the rise in the past ${timePeriod}. Great job ${nick}. Keep going.

adhavgtrdupp—Excellent job! Your adherence is on the rise over the past ${timePeriod}. Keep it up.

adhavgtrdupp—Your adherence score keeps rising, keep up the fantastic effort. Testing as often as this will be beneficial.

adhavgtrddwn—Don't let this downward trend in your adherence score continue. Take all your readings next time.

adhavgtrddwn—You're adherence score is trending down over the past ${timePeriod}. Turn it around by taking all your readings.

### 100% Current Adherence Score.

One portion of this rule applies when the current submission adherence percentage is 100% and the previous two submissions were for less than 100%. Another portion applies if the 14-day adherence score reported to the user is 100%

Application: type 1 and type 2

Circumstance codes: adhavgcur100 and adhavg14d100

Parameters: none

Priority: Least Recently Seen

### Messages:

adhavgcur100—Good work on improving your regimen adherence ${nick}.

adhavgcur100—Great work on getting your current adherence up to 100% ${nick}.

adhavg14d100—You've done a fantastic job taking your prescribed readings ${nick}.

100% for the last two weeks!

adhavg14d100—Your two week adherence score is 100% ${nick}. Great work!

### Poor Adherence and No Morning/Evening Readings.

This rule requires that the adherence percentage for each of the last three days is less than 100% but greater than 0% and that over the last three days no readings were submitted with a time of day before 11 am or no readings were submitted with a time of day after 6pm.

Application: type 1 only

Circumstance codes: mismrn and miseve

Parameters: none

Priority: Least Recently Seen

### Messages:

mismrn—You seem to keep missing your morning reading. You know it's important, try to make it happen. You know it's important, so try to make it happen.

miseve—You seem to keep missing your evening reading. Please try to make time to take it every evening.

Not Submitting Daily.

One portion of this rule looks at the last two weekends to see if they have not submitted on any of the four days of the weekend. It is also required that the patient submitted at least once between the two weekends. This portion of the rule runs only on the first submission after a weekend.

Another portion of this rule looks to see if the patient has consistently submitted every other day or less over the last week. It requires that they submitted today, but not yesterday, and that they submitted only two additional times in the five days prior to that.

Application: type 1 and type 2

Circumstance codes: missbmwkd and missbmreg

Parameters: none

Priority: Least Recently Seen

### Messages:

missbmwkd—You didn't submit readings for the second weekend in a row. Try to submit your readings every day, including on the weekends.

missbmreg—Submitting every day will help you monitor your diabetes. Try not to miss any days and bring your adherence average up.

missbmreg—Daily reading submission will help give you perspective on how you are doing everyday, and it helps me help you.

### Adherence Numbers.

This rule takes care of calculating the average adherence numbers that the Collector needs for displaying the graphical response screen and the adherence graph. The 14-day adherence score is calculated for the previous 14 days from yesterday. The current adherence score is calculated for the time between the last submission and the current submission. The weekly adherence scores are calculated as the average from the current day back seven days and then continuing back another seven days and another seven days, etc for a total of 6.

Application: type 1 and type 2

Circumstance codes: adhavg, adhwek

Parameters: val, curavg, 14davg, arwcol, arwdir

Priority: Always Send

### Miscellaneous Glucose Rules.

All Green Arrows.

This rule applies if the current average and current adherence percentage are better than the 14-day values.

Application: type 1 and type 2

Circumstance codes: allgrn

Parameters: none

Priority: Least Recently Seen

### Messages:

allgrn—Great job ${nick}! You've improved both your glucose average and your adherence average.

allgrn—With two green arrows, you're on the right track. Keep up the good work

${nick}!

allgrn—Nice work ${nick}. Your current glucose average and adherence average are better than your prior 14 day averages.

### Happy Birthday Message.

This rule applies if the current day is the patient's day of birth.

Application: type 1 and type 2

Circumstance codes: hpybdy

Parameters: none

Priority: Override Priority

### Messages:

hpybdy—Happy Birthday ${nick}!

30, 90 Days with the System.

This rule applies to the first submission after or on the patient's 30^{th} or 90^{th} day on the System. The patient must have submitted at least half the number of submissions for the time period.

Application: type 1 and type 2

Circumstance codes: dysonn030, dysonn090

Parameters: none

Priority: Override Priority

### Messages:

dysonn030—Congratulations on one month using the system. Your hard work is paying off for you.

dysonn100—Congratulations on three months using the system. Way to stick with it!

New to the System.

In the first week that a patient is on the System, this rule will look for encouraging or helpful things to say to new patients.

The first part looks for if they have missed submissions in the first week. Missing submissions is defined as having submitted at least one day, then not submitted at least one day, and then submitted again. The final submission must have occurred on or before the 7^{th} day on the system.

The second part of this rule looks for if they are submitting every day as required and their current adherence percentage is 100. This rule can occur on the patient's 3^{rd} through 7^{th} day on the system but can only be found one time.

The third part of this rule looks for if they are submitting every day as required, but their current adherence percentage is less than 100. This rule can occur on the patient's 3^{rd} through 7^{th} day on the system but can only be found one time.

Application: type 1 and type 2

Circumstance codes: newmissbm, newgodjob, newmisrdg

Parameters: none

Priority: Override Priority

### Messages:

newmissbm—Hello ${nick}. So far you've been doing well. It is important, though, that you submit your readings at least once a day. Keep up the good work!

newmissbm—Hi ${nick}. Please do your best to submit your readings at least once a day.

newgodjob—${nick}, with just a few days on the program, you've demonstrated a firm commitment to maintaining your glucose measurement regimen. Great job and keep up the good work!

newgodjob—Excellent start! 100 percent adherence for the first week is great. See you tomorrow!

newmisrdg—Hi ${nick}. You're doing a great job submitting your readings every day. Your next step is to take the number of glucose measurements per day recommended for you. You're doing well, keep up the good work!

newmisrdg—We received at least a reading everyday, nicely done. We hope to see all your readings for soon.

### Successful Submission.

This rule generates the circumstance code every time. It is a fallback rule to make sure something is generated.

Application: type 1 and type 2

Circumstance codes: thxsbm

Parameters: none

Priority: Least Recently Seen

### Messages:

thxsbm—Thanks for submitting your readings, keep it up!

Weight and Blood Pressure Adherence Rules.

### Non-compliance.

This rule simply checks the regimen and the readings submitted and sees if the patient is compliant. For a once a day regimen this rule is triggered if there is a missing reading on any completed day between their last submission and this submission.

For weekly regimens it is triggered if they have not submitted the required number of readings for the last 7 days, but not including today. It does not get triggered if there is a new reading for today.

Application: type 1 and type 2

Circumstance codes: noncmpday, noncmpwek

Parameters: readingType

Priority: Always Send

### Messages:

noncmpday—You missed a ${readingType} reading, don't forget to take all your readings every day.

noncmpwek—You're missing a ${readingType} reading in the past week. Don't forget to take your ${readingType} reading ${regimen}

Missing Today.

If the patient submits readings of another reading type and has not submitted for this reading type, then a circumstance code is generated as a reminder to check. For instance, they submit their glucose readings in the morning but do not include their BP which they are suppose to check daily. This rule then applies.

For weekly regimens if they would be out of compliance for not taking a reading on the current day, then the reminder is generated.

Application: type 1 and type 2

Circumstance codes: remtdyday, remtdywek

Parameters: none

Priority: Always Send

### Messages:

remtdyday—Don't forget to submit your ${readingType} reading today.

remtdyday—Did you forget to take your ${readingType} reading? You're suppose to take it every day.

remtdywek—${nick}, it's your day to submit your weekly ${readingType} reading, don't forget.

### Messages.

This section describes the Server's conversion of circumstances codes into a message for the patient. The first step is choosing which circumstance code to use and the second step is selecting which message to send for that circumstance code.

### Circumstance Code Selection.

The output of the Data Analysis algorithm is zero or more circumstance codes. The message selection process takes those circumstance codes and the history of what has been sent to the patient and determines which circumstance code to use this time. Choosing the circumstance code to use involves two priority rules. Each circumstance code above was assigned one of the priority rules: override and least recently seen.

If one or more generated circumstance codes are assigned priority override then each of those circumstance codes is used. This is the only case where a patient would receive more than one message from the server.

If there are no priority override circumstance codes generated then the least recently seen priority takes effect. If only one circumstance code was generated then obviously it is used. If more than one circumstance code is generated then the code least recently seen by the patient is used. If there are two circumstance codes that have never been seen by the patient then the system picks the one that was generated first.

### Message Rolling.

The second part of determining which message to send is to take the chosen circumstance code and pick which text message to send. A patient who sees the same computer-generated message repeatedly may come to discount it as information. The server stores multiple messages for many medical circumstances. This allows the system to reiterate the meaning without delivering the same message the user received before.

Once a medical circumstance is found, if it is found again then the next message in the sequence is sent. Once the last message for the sequence is sent the system starts back at the beginning with the first message. The number of messages available for each circumstance code is variable and codes that are expected to occur most frequently can have the largest number of messages.

### Message Variable Substitution.

Messages can contain variables that are substituted with the correct value at runtime, so that situation-specific data such as the number of missing readings or a number of days can appear in a message. The format is ${variable}.

nick—the patient's nickname or first name if no nick is specified

regimen—the regimen frequency value and count (e.g., 2 times daily)

patient—the patient's full name (first and last)

clinician—the clinician's full name (first and last)

guardian—the guardian's full name (first and last)

currentDate—the current date in the locale specific format

currentDateTime—the current date and time in the locale specific format

timeOfDay—the label for the time of day that the message is for: 5 am-10 am (morning), 10 am-2 pm (mid-day), 2 pm-5 pm (afternoon), 5 pm-9 pm (evening), and 9 pm-5 am (nighttime).

timePeriod—last week, last two weeks, last month

### Notifications.

Notifications are used to send unsolicited information to the patients, guardians and clinicians. For patient's this would be a reminder to submit readings. For guardians and clinicians it is information about one of their patients. Guardian and Clinician Notifications.

Notification occurs immediately after the patient submits data or at the scheduled reminder hour for the patient. A patient can have more than one notification attached to them such that multiple clinicians and one guardian could all be notified about different events related to the same patient.

The following list describes the type of notifications that can be specified:

If the patient has not submitted readings in X days.

If the patient submits a reading above X.

If the patient submits a reading below X.

### Message Duplication.

Guardians or clinicians can also choose to receive every message that the patient receives. This option overrides any notifications they have configured. PatientUpload Reminder.

The System stores an hour of the day chosen by the user and stored in his profile; at this time of day, the system determines whether he has sent in data readings in that day. If he has not, the system sends an email message to the patient's phone as well as to their personal email address if they entered one reminding them to submit their readings. The message sent to the patient will be less than 160 characters, the maximum length allowed for SMS on, for example, the AT&T network.

At the same time the system also checks to see if the guardian or clinicians should be notified about patient adherence. Templates.

The Server utilizes templates during the patient creation process. An overall patient template contains patient-level information and may contain zero or more notification templates and zero or more regimen templates.

Regimen templates contain information about what type of reading the patient should collect, how often, and provides some value inputs used by the data analysis rules.

Each notification template is for a specific reading type and specifies when the user (guardian or clinician) would be notified about compliance or reading values.

### Aborted Data Submissions.

When the patient submits readings the readings remain stored on the phone until the response is received by the phone which provides acknowledgement that the readings have been stored on the server. If the patient aborts the submission before receiving a response then patient is able to use the “Send Stored” option to submit the readings later or if they do a “Collect and Send” at a later time the old readings are sent as well. On the server side this means the server may receive the same readings twice. If the server receives readings which are all duplicates it does not need to redo analysis and message selection. It can instead simply resend the last message as this would only occur if the patient aborted the submission process before receiving the response. If new readings are sent along with some old, previously submitted readings, then a new analysis is performed and a new message chosen and sent back.

### Server User Interface.

The Server UI (User Interface) runs on the same server as the Server Analysis and Messaging component. Access to the Server UI is over https (SSL) only and requires a username and password to access the site.

The Server UI will support multiple simultaneous languages based on the user's web browser locale setting.

### Server UI Introduction.

This section explains the user interface of the Server UI. The focus of this document is on detailing the pages and the navigation among them.

### Navigation.

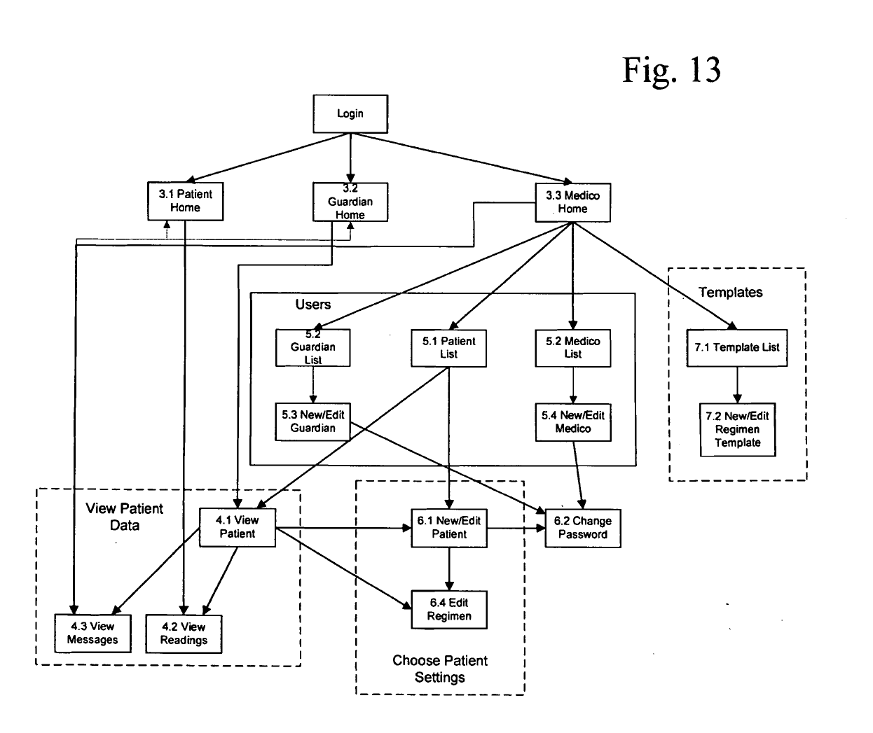

The following diagram of FIG. 13 shows the pages described here and the navigation among them. Each arrow represents a possible option for a user to go from one page to another on the website.

Website Navigation is illistrated and described with reference to FIG. 13 as follows:

### Roles.

The system contains three different roles; patient, guardian, and clinician that control what a user can see or do on the Server UI. Patients can view their data and change their profile. Guardians can view their patient's data, change their pSatient's profile, and change their own profile. Clinicians can do all features.

### Login Screen.

The Login page allows entry of username and password. A Login button submits the request. If login is successful, the system displays the home page corresponding to the type of user logging in. If the login fails, the Login page is shown again with an error message.

### Common Features.

Each page (except the login page) contains a common banner at the top with links: Home, My Profile, and Logoff.

### Home Pages.

Each of the four user types sees a different home page after they login. Three home pages exist, one for each user type: patient, guardian, and clinician.

### Patient Home.

The Patient home page is the highest visibility page as this is where each patient will start after logging in. The page contains the following parts:

The patient name and a welcome message along with the patient's clinicians' names and guardian's name

The latest Adherence Score and average glucose value

The last ten readings sent in

The last 5 messages sent in

A list of the regimens prescribed for the patient

Links to the View Readings Page, View Recent Readings Chart and the View Messages Page

### Guardian Home.

The Guardian home page displays the guardian's name and a welcome message. It also displays the most recent messages sent to the guardian plus a link to view all messages sent to the guardian. Next is a drop-down list of patients; selecting one from that list displays the Patient View Page for that patient.

### Clinician Home.

The Clinician Home page displays the clinician's name and a welcome message. If the clinician receives messages about any of his patients, it also displays a list of the most recent messages, and a link to go to the full list of messages. Following this are links to the Patient List, the Guardian List, the Clinician List, and Manage Templates page.

Additionally a link exists to send all of the Clinician's patients a message.

Next is displayed system report information:

total patient count

active patient count

global adherence score

the total number of readings submitted

total number of submissions

### List Pages.

Many of the pages display lists of objects in tabular form and use a common paging and sorting mechanism. The paging control allows the user to do the following:

Choose how many rows to show on a page.

Move to the next and previous pages.

Move to a specific page.

Sort the table by (some) columns.

Many pages or columns support sorting. Sorting is done by clicking on the column header. A small arrow in the header of a given column indicates whether the results are currently sorted up or down by that column and clicking the currently sorted column reverses the sort order. A default sorting order and column exist for each table.

### Patient List.

The Patient List provides a list of patients in a table with the following columns: patient name, clinician name, date of last submission, 14-day adherence average, 14-day glucose average, number of high readings in last 7 days, number of low readings in last 7 days, and patient's account status.

The table can be sorted by any column. Each patient name is a link to the View Patient page. Below the table is a link to create a new patient.

Guardian and Clinician Lists.

The Guardian and Clinician List pages provide a list of Users in a table with the following columns: username, first name, last name, role, and user type. The table allows sorting by any column. Each username is a link to the Edit User page. Below the table is a link to create a new user.

### Patient Template List.

The Patient Template List provides a list of Patient Templates in a table with the following columns: name, description, and options. The options column has one option to remove the Patient Template. The table allows sorting by name. The name column is a link to the New/Edit Patient Template page. Below the table is a link to the New Patient Template page.

### View Readings.

The View Readings page shows a table of readings with four columns: date, reading type, value, and unit of measure. The table can be sorted by any of the columns.

### View Messages.

The View Messages page shows a table with columns: sent time, delivery method, and the message text. The table can be sorted by sent time and delivery method. For patient messages it also shows the two arrows and average values the same as displayed on the phone.

### New/Edit Pages.

Numerous pages contain input components for creating or modifying an entity. These pages are referred to as New/Edit Pages. These pages are similar in layout to each other, with input components left justified and with Save and Cancel buttons to the right. Clicking the Save button submits the new or modified information to the server. If any errors occur, control is returned to the New/Edit Page with the error messages shown at the top of the page. A save with no errors, or clicking the cancel button, returns the user to the previous page.

### New/Edit Patient Page.

This page allows creating and modifying patient profiles. It can be accessed only by clinicians.

The following input text fields exist:

Username—(30 chars) must be unique across all user types in the system. This field is only visible when creating a new patient.

Password—(60 chars) this field is only visible when creating a new patient.

First name—(50 chars)

Last name—(50 chars)

Nick name—(25 chars)

Email address—(100 chars), must have an @ sign

Cell phone number—(20 chars)

The following drop-down lists exist:

Role

Default language

Clinician

Guardian

Date of Birth—month, day, year

Sex

Reminder hour

Cell phone carrier

Cell phone type

The following checkbox input fields exist:

Allow clinician notifications

Disable account (only for edit)

### New/Edit Guardian Page.

The New/Edit Guardian page contains some of the same fields defined above for the patient: username, password, first name, last name, email address, role, receiver category, default language, and disabled account flag. Additionally, there is a choice between receiving duplicates of the patients or clinicians messages, or no messages at all.

When editing an existing guardian a link to change the guardian's password exists.

### New/Edit Clinician Page.

The New/Edit Clinician page contains some of the same fields defined above for the patient: username, password, first name, last name, email address, role, receiver category, default language, and disabled account flag.

When editing an existing clinician a link to change the clinician's password exists.

### New/Edit Regimen.

The New/Edit Regimen page contains the following text fields and drop down lists for creating or modifying a patient's regimen:

Condition drop down list—for selecting the condition to which this regimen corresponds

Reading Type drop down list—for selecting the reading type to which this regimen corresponds

Frequency drop down list—for selecting the frequency of the readings, daily or weekly

Value drop down list—for entering selecting the number of readings required per frequency period

Condition/Reading Type specific parameters
Diabetes/Glucose—target low, target high, danger low, danger high—all integers

Device Model and Bluetooth ID—to specify the type of device the user has and the Bluetooth ID for the system Connect in order to simply patient configuration of the phone.

### New/Edit Notification.

The New/Edit Notification page contains the following text fields and drop down lists for creating or modifying a notification:

No Submission—specify the number of days with no submissions that results in a notification

High Reading—specify a glucose value at or above which results in a notification

Low Reading—specify a glucose value at or below which results in a notification

### Patient Template Edit.

The Patient Template Edit page contains text fields for name and description and contains drop down boxes to set the default clinician, the default receiver category, and the default reminder hour.

### Regimen Template Edit.

The Regimen Template Edit page contains the same input fields as the Regimen page specified above.

### Notification Template Edit.

The Notification Template Edit page contains the same input fields as the Notification page specified above.

Other Pages.

View Patient.

The screen to view patient data contains the following summary of patient data:

The patient's detail information
name
email address
clinician
default language
condition
birth date
sex
start date (of this patient in the system)
clinic
guardian
account status
cell phone carrier, number, and phone type
last submission date

A link to edit the patient's information

A link to change the password (Clinicians only)

A link to enable/disable the account (Clinicians only)

Patient statistics for their adherence and reading values

Each Regimen specified for this patient with links to edit, remove and add a new regimen

Adherence score

A link to the scatter graph of the patient's recent readings

A link to add or edit the notification rule for this patient

The most recent readings reported to the Server

The most recent messages sent from the Server

For Clinicians an option exists to send the patient a message

For Clinicians an option exists to resend the Collector installation invitation

### User Profile.

The User Profile page is accessible from the top navigation bar. It provides access to the current user's data that they can modify: name, email address, default language, and for patients, upload reminder time.

On the Guardian user's profile page is also a selection component to choose whether to receive messages that duplicate the patient's messages or the clinicians' messages.

Guardian user's can also access their patient's profile page in order to modify their patient's settings.

### Send Message.

This page allows a clinician to type in a message to be sent to one patient or all of their patients. The message length is limited to 160 characters on the , for example, AT&T network as the message is ultimately delivered to the phone as an SMS message.

### Change Password.

When a user changes his/her own password, he/she must enter the old password and the new password twice; the password entered is masked so a casual observer cannot see what it is while being entered. Guardians and Clinicians may also change their patient's password which does not require entering the old password.

Having thus described the invention, the same will become better understood from the appended claims in which it is set forth in a nonlimiting manner.

What is claimed is:

1. A system for monitoring a patient's health and providing feedback to improve self-care and regimen compliance, comprising:
a medical data collection device for collecting medical data from a patient;
a data transmission device associated with the medical data collection device for transmitting the data to a backend system; and
a backend system programmed for receiving said data and generating a feedback message to the patient.

2. The system for claim 1, further comprising a collector device which is a Bluetooth capable wireless telephone for receiving the data from the medical collection device.

3. The system of claim 1, wherein said backend system comprises a server programmed for collecting medical measurement data from the collector device and for sending messages to at least one of a user, guardian for a minor user and a clinician for the user.

4. The system of claim 1, further comprising a profile data store at the backend system containing information about a user, medical parameters being measured, parametric data, and data for making decisions about feedback messages to be delivered.

5. The system of claim 1, wherein said medical data collection device is a glucometer, and said backend system is configured for provided feedback to address diabetics.

6. The system of claim 1, wherein said medical data collection device is a blood pressure meter, and said backend system is configured for providing feedback to address high blood pressure.

7. The system of claim 1, wherein said medical collection device is a scale, and said backend system is configured for providing feedback to address an overweight condition.

8. The system of claim 1, wherein the backend system is further configured for receiving multiple types of medical data for a patient and for generating feedback messages based on the multiple types of medical data received.

9. The system of claim 1, where the backend system is programmed for delivering different types of messages dependant on whether a patient is an adult, teenager, a juvenile, a man or a woman.

10. The system of claim 1, wherein the collector device is programmed for having collected medical data persistently stored on the collector device after transmission to the backend system until acknowledgement of receipt at the backend system is received.

11. A method of monitoring patient health and providing feedback to improve self-care and regimen compliance, comprising:
collecting medical data from a patient with a medical device;
transmitting said data to a backend system; and
analyzing the data with the backend system and transmitting a feedback message based on said analysis.

12. The method of claim 11, wherein said data is transmitted to a collector device capable of storing the data and transmitting the data to the backend system, and further comprising transmitting the data to the backend system from the collector device and receiving the feedback message at the collector device.

13. The system of claim 11, wherein said medical device is a glucose meter and said feedback message relates to diabetes treatment.

14. The method of claim 11, wherein said medical device is a blood pressure monitor and said feedback message relates to treatment of high blood pressure.

15. The method of claim 11, wherein said feedback message is tailored to specific characteristics of the patient.

16. The method of claim 11, wherein said backend systems includes patient information used in conducting the analysis.

17. The method of claim 11, wherein different types of medical data is collected, and the analysis is conducted based on the different types of data collected.

18. The method of claim 11, further comprising transmitting the feedback message to a guardian when the patient is a juvenile.

19. The method of claim 11, wherein the message created determined based on whether the patient is a juvenile, teenager, adult, male or female.

20. The method of claim 11, wherein data about a patient's psychological profile is used to generate the feedback message.
